(19) United States
(12) Patent Application Publication
(10) Pub. No.: US 2013/0012807 A1
(43) Pub. Date: Jan. 10, 2013

(54) SYSTEM AND METHOD FOR TRACKING POSITION OF HANDHELD MEDICAL INSTRUMENTS

(75) Inventors: FOUAD AL-SUNNI, DHAHRAN (SA); MOUSTAFA ELSHAFEI, DHAHRAN (SA)

(73) Assignee: KING FAHD UNIVERSITY OF PETROLEUM AND MINERALS, DHAHRAN (SA)

(21) Appl. No.: 13/176,691

(22) Filed: Jul. 5, 2011

(51) Int. Cl. A61B 5/05

(52) U.S. Cl. 600/424

(57) ABSTRACT

The system and method for tracking the position of handheld medical instruments provides for instantaneous feedback and instruction to a medical practitioner during performance of a medical procedure. The system and method utilize a graphical user interface, which displays data related to at least a portion of a patient's body. The user then selects a body part of the patient for performing a selected medical procedure. A plurality of pulse receivers are provided for detecting and receiving very narrow pulse electromagnetic pulses. A plurality of instrument pulse emitters are mounted on a handheld medical instrument for selectively transmitting first very narrow pulse electromagnetic pulses, and a to plurality of patient pulse emitters are positioned on the selected body part of the patient for selectively transmitting second very narrow pulse electromagnetic pulses. The position and orientation of the handheld medical instrument with respect to the selected body part is then determined.

### BACKGROUND OF THE INVENTION

1. Field of the Invention

The present invention relates generally to medical sensors and imaging systems, and particularly to a system and method for tracking position of handheld medical instruments, e.g., sensors and imaging devices, with respect to a selected patient body part.

2. Description of the Related Art

Range finding techniques are known in the art. Such range finders often include generation of an electromagnetic or ultrasonic pulse, and the range to a target is determined based upon the time difference between transmission of the pulse and reception of a reflection of the pulse. Such techniques, however, typically do not have the accuracy required to also produce accurate measurements of the orientation of a particular angle (i.e., roll, yaw and pitch). For medical procedures, the orientation of a patient's body part and the orientation of the medical instrument applied to the body part are obviously critical. Thus, conventional positioning techniques may not be easily applied to medical procedures.

Similarly, orientation measuring techniques are known, including the use of gyroscopes and complex optical scanning techniques. Such techniques, though, require the use of complex and often heavy equipment, which cannot be easily arranged either on or near a delicate medical instrument (such as a scalpel or probe, for example). It would be desirable to provide a non-intrusive and easily established position and orientation detection system to provide feedback and instruction to a medical practitioner during medical procedures.

Thus, a system and method for tracking the position of handheld medical instruments solving the aforementioned problems is desired.

### SUMMARY OF THE INVENTION

The system and method for tracking the position of handheld medical instruments provides for instantaneous feedback and instruction to a medical practitioner during use of a handheld medical instrument, e.g., a sensor, an imaging device, an ultrasonic scanning unit, a surgical instrument, etc. The system and method utilize a graphical user interface that displays data related to at least a portion of a patient's body. The user then selects a body part of the patient for performing a selected medical test, imaging scan, or procedure.

A plurality of pulse receivers are provided for detecting and receiving very narrow pulse electromagnetic pulses. A plurality of instrument pulse emitters are mounted on a handheld medical instrument for selectively transmitting first very narrow pulse electromagnetic pulses, and a plurality of patient pulse emitters are positioned on the selected body part of the patient for selectively transmitting second very narrow pulse electromagnetic pulses.

The position and orientation of the handheld medical instrument with respect to the plurality of pulse receivers is determined based upon travel time between transmission of the first very narrow pulse electromagnetic pulses and detection thereof. Similarly, a position and orientation of the selected body part with respect to the plurality of pulse receivers is determined based upon travel time between transmission of the second very narrow pulse electromagnetic pulses and detection thereof. From this information, the position and orientation of the handheld medical instrument with respect to the selected body part may be determined based upon the position and orientation of the handheld medical instrument with respect to the plurality of pulse receivers and the position and orientation of the selected body part with respect to the plurality of pulse receivers. User feedback is then provided to the medical practitioner via the graphical user interface based upon the selected medical procedure and the position and orientation of the handheld medical instrument with respect to the selected body part.

These and other features of the present invention will become readily apparent upon further review of the following specification and drawings,

### Brief Description of the Drawings

FIG. 1 is a diagrammatic overview of a system for tracking the position of handheld medical instruments according to the present invention,

FIG. 2 is a perspective view of an exemplary handheld medical instrument used with the system for tracking the position of handheld medical instruments according to the present invention.

FIG. 3 is a block diagram of a controller and timing unit in a system for tracking the position of handheld medical instruments according to the present invention.

FIG. 4 is a block diagram of a pulse emitter in a system for tracking the position of handheld medical instruments according to the present invention.

FIG. 5 is a schematic diagram of the pulse generator of the pulse emitter of FIG. 4,

FIG. 6A is a schematic diagram of a tunable delay cell of the pulse generator of FIG. 5.

FIG. 6B is a schematic diagram of a reference cell of the pulse generator of FIG. 5.

FIG. 7 is a waveform diagram showing the generation of narrow pulses through adjustment in delays in the pulse generator of FIG. 5.

FIG. 8 is a block diagram of a pulse receiver in a system for tracking the position of handheld medical instruments according to the present invention.

FIG. 9 is a diagram illustrating an exemplary pulse position coding sequence in a system and method for tracking position of a handheld medical instrument according to the present invention.

FIG. 10 is a block diagram illustrating functionality of the pulse receiver of FIG. 8.

FIG. 11 is a diagrammatic front view of a display screen showing an exemplary graphical user interface in a system for tracking the position of handheld medical instruments according to the present invention.

Similar reference characters denote corresponding features consistently throughout the attached drawings.

### DETAILED DESCRIPTION OF THE PREFERRED EMBODIMENTS

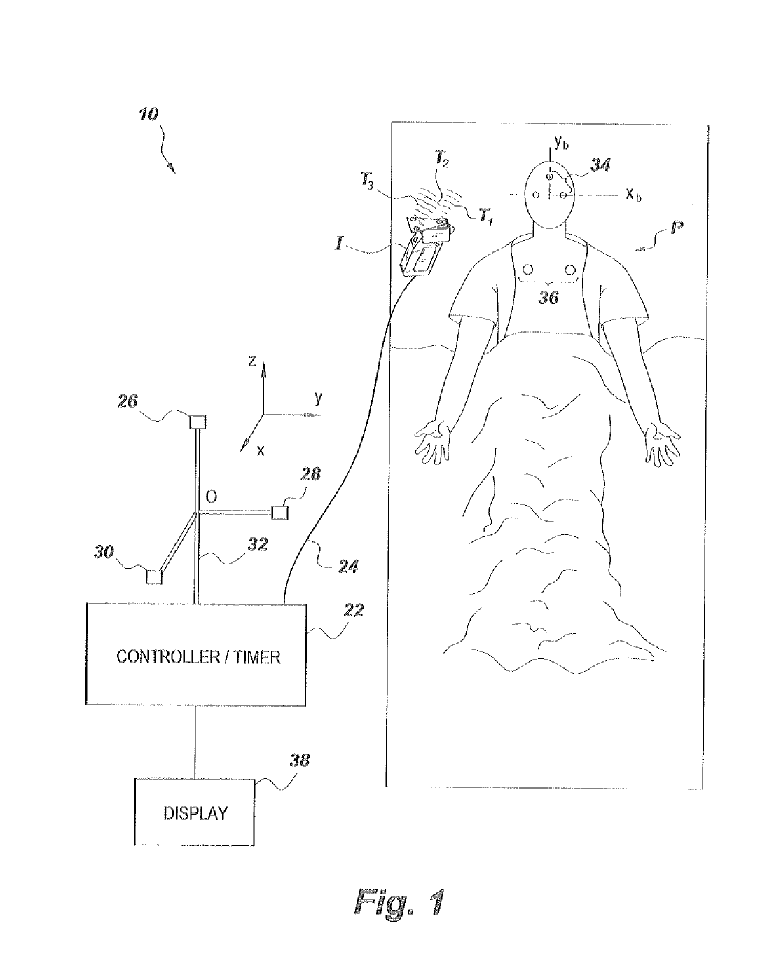

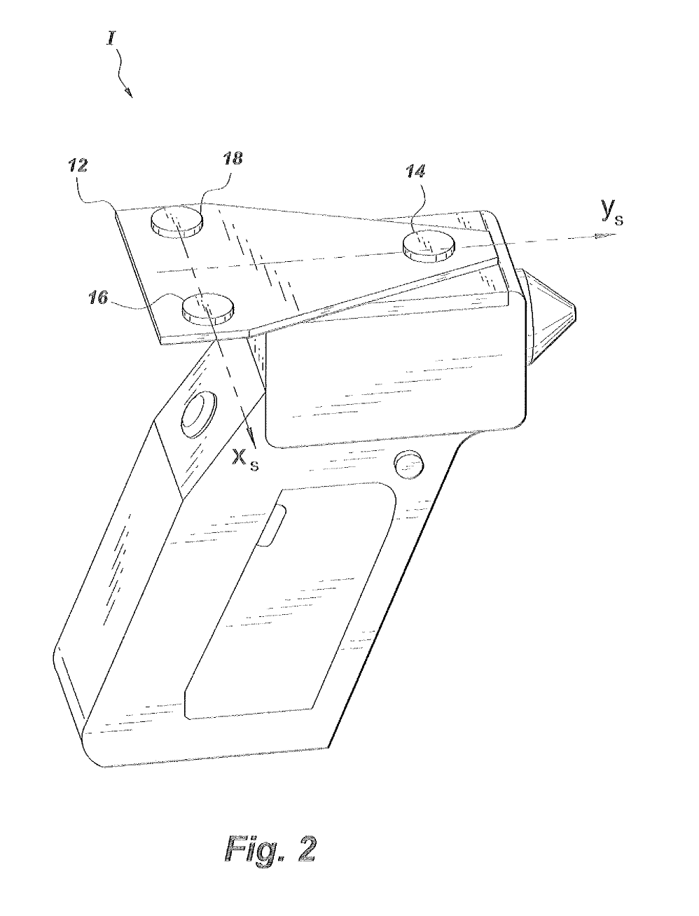

FIG. 1 illustrates an overview of the system for tracking the position of handheld medical instruments 10, such as exemplary instrument I. As best shown in FIG. 2, a support, such as mounting plate 12, is fixed to the instrument I for supporting three pulse emitters 14, 16, 18. It should be understood that the mounting plate 12 may have any desired overall shape and relative dimensions, and the overall shape and relative dimensions depend upon the particular instrument I to which the mounting plate 12 is applied. The mounting plate 12 may be secured to the instrument I via any suitable type of fixture. The handheld medical instrument I may be any suitable type of medical instrument, for example, an ultrasonic scanning unit, a surgical instrument or the like. It should be understood that the instrument I illustrated in FIGS. 1 and 2 is shown for exemplary purposes only.

As shown in FIG. 1, the three pulse emitters 14, 16, 18 selectively transmit corresponding electromagnetic pulses T₁, T₂, and T₃. A controller and timing unit 22 is positioned near a patient P and communicates with the pulse emitters 14, 16, 18 via a communications cable 24, which may be a fiber optic cable or the like. Pulse receivers 26, 28, 30, which are also in communication with the controller and timing unit 22, are mounted on a support structure 32. In order to simplify descriptions in what follows regarding the particular method of transmission and reception, the received pulses (i.e., received by the pulse receivers 26, 28, 30) will be referenced as electromagnetic pulses R₁, R₂, and R₃, corresponding to transmitted pulses T₁, T₂, and T₃. At least one set of patient pulse emitters are positioned on selected sites on the patient's body. In FIG. 1, two such sets of pulse emitters 34, 36 are show respectively positioned on the patient's head and chest.

A display 38 is provided, the display 38 also being in communication with the controller and timing unit 22. The display 38 provides a graphical user interface that allows the user to select the part of the body to be examined. The display 38 preferably includes a touch screen or a similar input/interface device. The graphical user interface suggests certain preferred locations, based upon the particular medical examination and procedure, and the user preferably confirms his or her selection by touching the desired places on the screen. FIG. 11 illustrates an exemplary graphical user interface 40 shown on the display 38.

FIG. 3 illustrates the system components of the controller and timing unit 22, as will be described in greater detail below. The controller and timing unit 22 sequentially activates each of the pulse emitters (PEs) 14, 16, 18 via a sequence of encoded signals generated by a transmitter 42, which is sent via the communications cable 24. Each pulse emitter 14, 16, 18 receives the incoming coded signal sequence and converts the signal to a series of coded narrow pulses. The coded series of narrow pulses are then emitted as a sequence of ultra narrow pulses of electromagnetic radiation T₁, T₂, and T₃. As noted above, the pulse receivers 26, 28, 30 (mounted on orthogonal axes provided by the support 32, as shown in. FIG. 1) receive pulses R₁, R₂ and R₃, corresponding to the transmitted pulses T₁, T₂, and T₃.

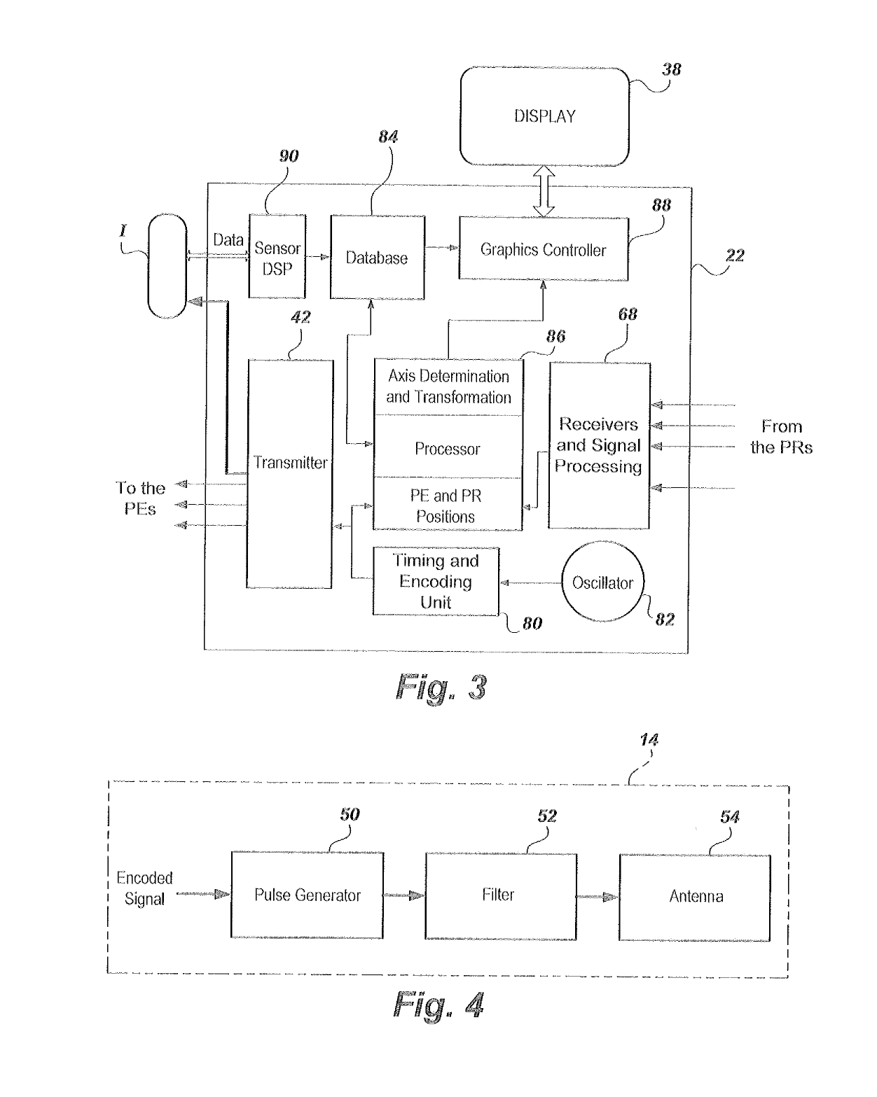

As will be described in greater detail below, the system and method for tracking the position of the handheld medical instrument utilizes very narrow pulse (VNP) transmission for range determination. VNP is carrier-less; i.e., data is not modulated on a continuous waveform with a specific carrier frequency, as in narrowband and wideband technologies. Carrier-less transmission requires fewer radio frequency (RE) components than carrier-based transmission, as shown in FIG. 4. FIG. 4 illustrates the basic components for the pulse emitters 14, 16, 18. The encoded signal is transmitted from the controller and timing unit 22 via the communications cable 24 (and internally through the instrument 1 to the emitters 14, 16, 18 mounted on the support 12) to a pulse generator 50 of the pulse emitter. The pulse generator 50 generates the signal for transmission, which is passed through a filter 52, and then through an antenna 54 for transmission. This greatly simplifies the transmission process, requiring only three basic components since the pulse emission is not based on a modulated radio carrier frequency.

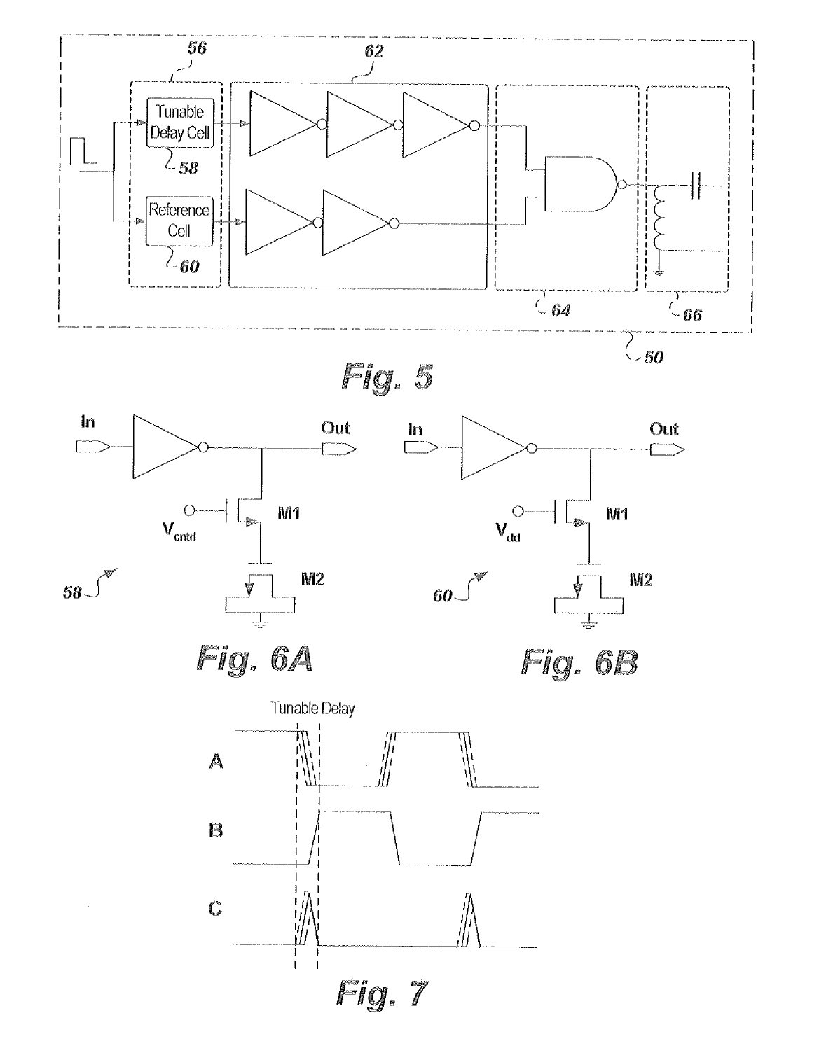

FIG. 5 illustrates the pulse generator 50. The pulse generator 50 includes a tuning delay circuit 56 (which includes a tunable delay cell 58 and a reference cell 60), an inverter block 62, a NAND-gate block 64, and a pulse shaping circuit 66 using shunt-capacitor delay elements, all preferably formed in a single chip. The inverter block 62 and the NAND-gate block 64 together form an impulse-forming circuit, which serves as a receiver for the encoded signal from the controller 22 and a square wave source, which the pulse shaping circuit uses to generate the VNPs. The chip may be fabricated using the CMOS process at 0.25 or 0.18 μm. A single 2.5 V supply voltage may be used for the entire circuit.

Each pulse emitter obtains the encoded signals from the controller via the communication cable 24, which may be a fiber optic cable, coaxial cable or the like, and the encoded signal passes through the pulse generator unit 50, which then produces a corresponding series of VNPs. The VNP series then passes through the filter 52 and is sent to the antenna 54 for transmission as signals T₁ (from emitter 14), T₂ (from emitter 16), and T₃ (from emitter 18). The filter 52 limits the energy of the pulses to a specified bandwidth. The antenna 54 is designed to meet the bandwidth requirements, and to generate omnidirectional radiation.

FIG. 6A illustrates the tunable delay cell 58, In FIG. 6A, variable control voltage V_{cntrl} is applied to the gate of MOSFET M1 to produce continuous delay variation. However, for the reference cell 60, shown in FIG. 6B, the gate voltage V_{dd} of its MOSFET M1 is fixed, and thus the time delay is constant and provides a reference position to the tunable delay cell 58.

The impulse-forming circuit includes an inverted delay stage formed by the inverter block 62 and the NAND-gate block 64. The NAND-gate block 64 generates an impulse-like signal and provides driving capability to the next stage. This impulse is capable of evoking the impulse response of the succeeding component to further produce a monocycle pulse (or other types of pulse waveforms, as needed for VNP systems). The last stage of the tunable monocycle pulse generator is the pulse-shaping circuit 66, which includes a shunt on-chip spiral inductor and series capacitor.

As shown in FIG. 7, the encoded input signal is divided equally into two paths. In path A, the encoded signal passes through the tunable delay cell 58 (shown as the top path), and in the other path (path 13) the encoded signal goes through the reference cell 60 (shown as the middle path). At the output of the tunable delay cell 58, a square-wave signal (shown in path A of FIG. 7) with very short rising and falling times is generated and functions as one of the inputs to the inverter block 62 and the NAND-gate block 64. At the output of the reference cell 60, a second square wave signal (shown in path 13 of FIG. 7), also with very short rising and falling times but delayed with respect to path A, is generated and functions as a second input to the inverter block 62 and the NAND-gate block 64. When the two inputs to the NAND-gate block 64 arc both at a low state (approximately 0 V), as shown in the timing of FIG. 7, i.e., when these two reversed square waves are fed to the NAND-gate block 64, a narrow impulse-like signal is generated at the output node of the NAND gate, as shown in path C of FIG. 7. The width of this impulse signal depends on the relative time delay between these two square-wave signals and their rising and falling edges. The impulse signal, therefore, can be easily generated with a continuously tuned duration.

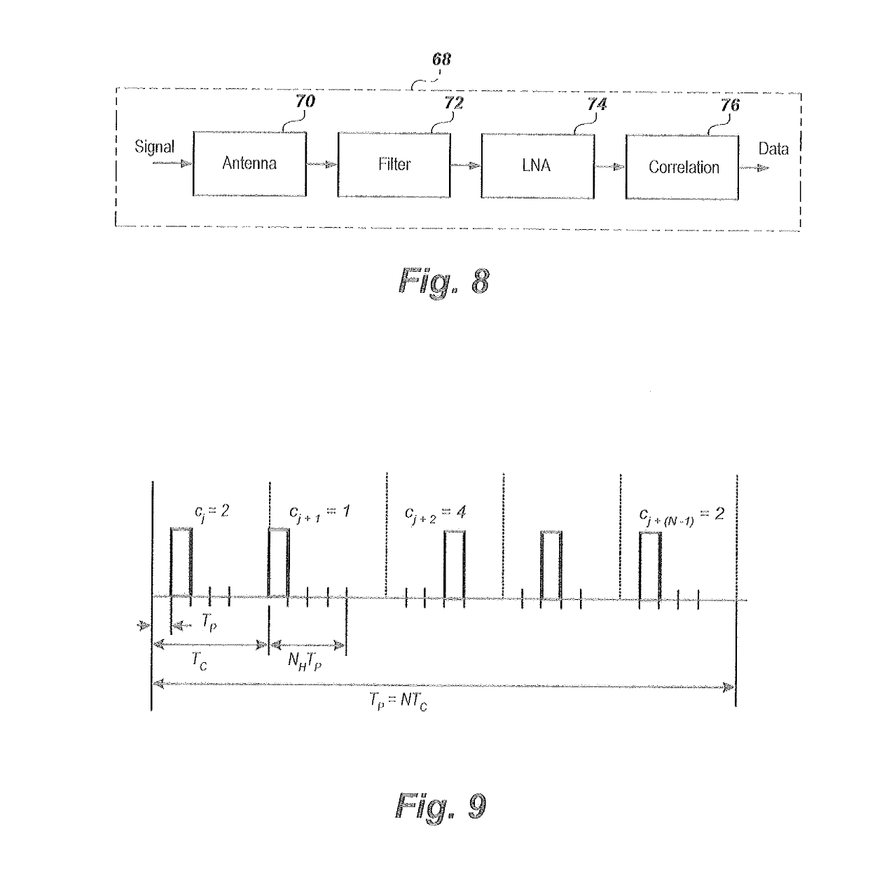

Referring to FIGS. 1 and 3, the pulse receivers 26, 28, 30 are connected to the controller and timing unit 22 by any suitable connection, such as a fiber optic cable or the like, and the received signals are processed by a very narrow pulse VNP receiver 68. The VNP receiver 68 is illustrated in FIG. 8. Each pulse receiver includes one or more wideband printed circuit board (PCB) miniature antennas 70. Two to four antennas may be used as a diversity antenna to reduce the effect of multi-path signals. The signal from the diversity antenna 70 is then passed to a band pass filter 72 to reduce the effect of the out-of-band noise. In the VNP receiver 68, the signal is then amplified using a low noise amplifier (LNA) 74, and is then passed to a signal correlator 76. FIG. 9 illustrates an exemplary pulse position coding sequence.

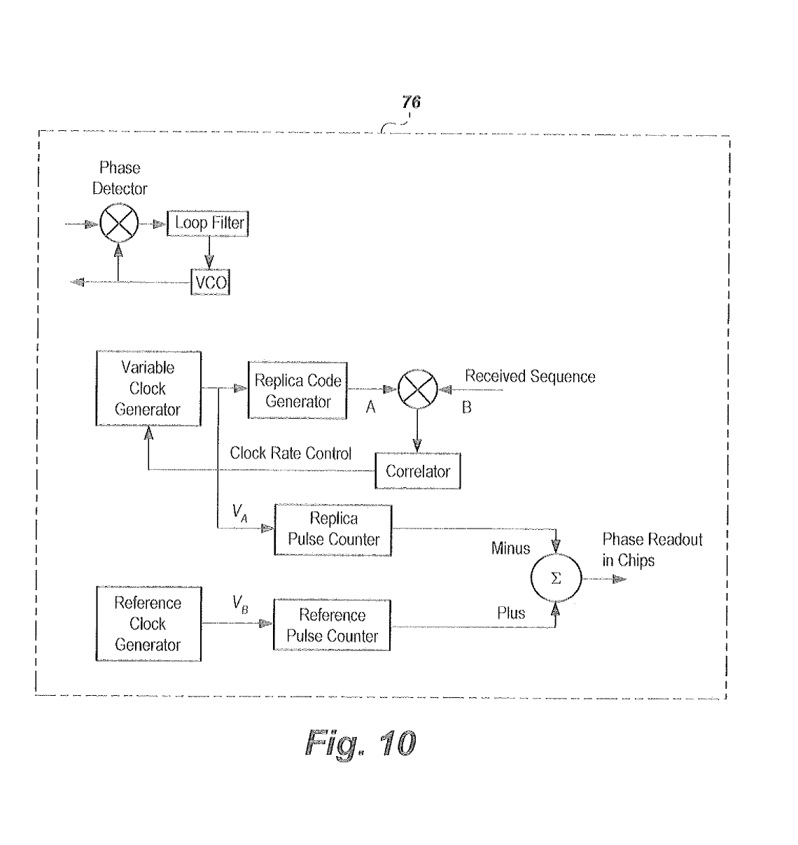

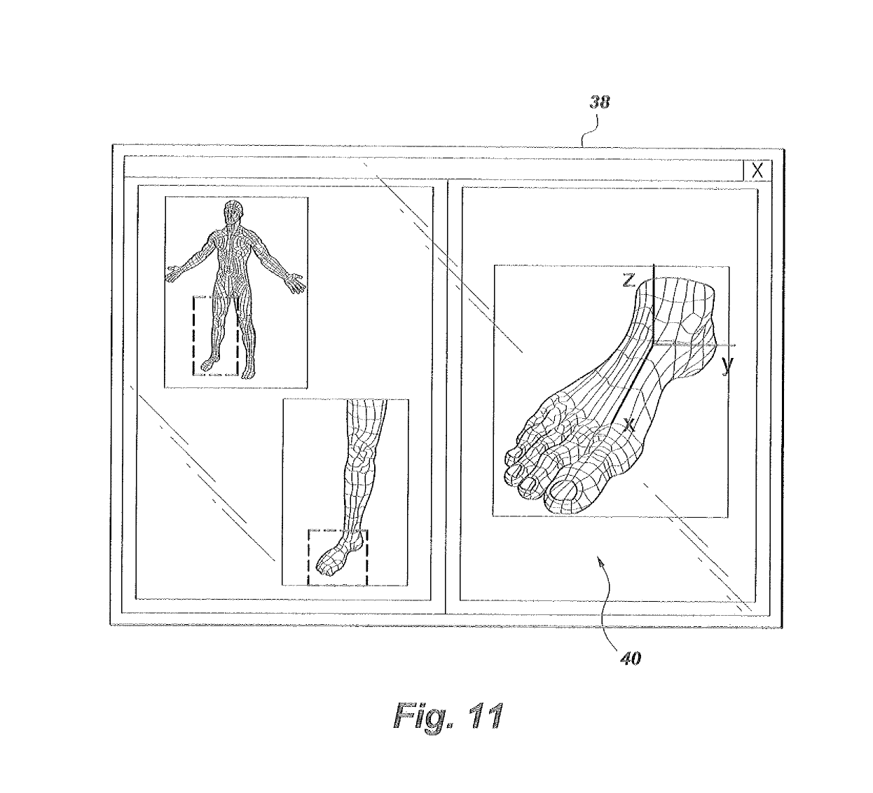

FIG. 10 illustrates the correlator 76 in detail. The correlator 76 involves two stages, including a course correlation stage to identify the time lag between the reference sequence-coded sequence of pulses and the received signal to within a chip period, and a fine registration stage to determine the delay between the received signal and the reference clock within a single chip period. In a preferred embodiment, a code sequence of 1,024 (Ts) pulses is used. In the following, the period of each chip is referred to as and the period of the pulse is given as Tₚ. The system 10 allows for precision registration within millimeters or sub-millimeters, it should be noted that such accuracy is not achievable by traditional localization methods using WiFi, RFID, etc. Each pulse receiver preferably includes a secondary tunable clock generator. The first received sequence of pulses are used to synchronize the secondary tunable clock generator, and the secondary tunable clock generator drives the correlator 76. Correlator 76 correlates the incoming sequence with a template sequence. Further, the clock count at which maximum alignment of the received sequence with the template sequence is detected and recorded, and this clock count is representative of the first time delay (i.e., the course delay time), and the second delay time is calculated as the phase difference between the master clock sequence and the secondary clock sequence (i.e., the fine delay time). The improved estimate of the time of arrival of the received sequence is obtained using the first delay time and the second delay time.

Referring again to FIG. 3, following processing within the receiver 68, the output is passed to a processor 86, which may be any suitable type of processor, such as that associated with a personal computer or the like, a microcontroller, a digital signal processor, or a programmable logic controller or the like. The processor 86 performs the calculations described below to calculate the pulse emitter and pulse receiver positions, along with rotation and axis calculations. The processor also controls the graphical user interface 40 displayed on the display 38 through a graphics controller 88, and is in communication with a database 84, which is stored in computer readable memory. A digital signal processor (DSP) 90 may also feed direct sensor data from the instrument I into the database 84.

In the present method, time of travel is first calculated. The transmitter sends a coded sequence of 1,024 pulses (or chip periods), and detection is performed using a matched Filter or a sliding correlator. The correlator determines the course time delay within one chip period. Fine difference is determined by the phase difference between a master clock 80, which is used in transmission, and a variable oscillator 82, which is used during correlation. The time of arrival tₐ is then given by:

tₐ=t_{coarse}+t_{fine}, (1)

where t_{course} represents signal travel time and t_{fine} represents fine difference correction. For an exemplary chip rate of 5 GHz, T_{c}=0.2 ns, and for a 1,024 chip code length, Tₚ=204.8 ns.

The physical spacing between the transmitter (i.e., pulse emitters 14, 16, 18) and pulse receivers 26, 28, 30 is typically between 60 cm to 2.0 meters. However adding the length of the cable(s) 24 and accounting for the lower speed of signal travel in the cable(s), the expected maximum length is about six meters, representing a total maximum delay between the transmitted and received signal of 20 ns. The measurement is preferably repeated sixteen times, and the average tₐ is calculated from these sixteen measurements.

In the preferred embodiment, a total of six transmission signals T₁, T₂, T₃, T₄, T₅ and T₆ are generated. Signals T₁, T₂, T₃ are respectively generated by pulse emitters 14, 16, 18 on instrument I, and pulses T₄, T₅ and T₆ are generated by patient emitter sets 34, 36 on the patient's body (representing the axes of the patient's body). Pulse receivers 26, 28, 28 are arranged on orthogonal Cartesian axes and have known locations with respect to a reference point O.

The time of arrival (TOA), given by tₐ, can be expressed as:

tₐ=t_{d}+t_{rec}+tₜᵣ, (2)

where t_{d} is the time of travel over the physical distance between the pulse emitter and the pulse receiver, t_{rec} is the receiver cable delay and processing delay, and tₜᵣ is the transmitter delay from the start of the code sequence to the transmitting antenna. For accurate distance measurements, both t_{rec} and tₜᵣ are measured and accounted for. Alternatively, the time difference of arrival (TDOA) may be used for better accuracy, as some of the sources of errors will be cancelled during the subtraction, such as the uncertainty in the transmitter delay.

For calibration purposes, the pulse emitters 14, 16, 18 are placed at a known location with known precise distances to the three pulse receivers 26, 28, 30. In the following calculation, the following convention for transmitted and received pulses is used. The true propagation time is t_{d,j}, where d represents the pulse emitter (i.e., pulse emitters 14, 16, 18 are referenced by d=1, 2, 3; respectively, and the patient pulse emitter sets 34, 36 are referenced by d=4, 5, 6, respectively) and represents the pulse receiver (i.e., pulse receivers 26, 28, 30 correspond to j=1, 2, 3). The calibration position is a holding position at a distance of μ cm from the reference origin O on the z-axis.

For three pulse receivers and six pulse emitters, there are a total of nine unknowns to be determined. Each pulse emitter is placed in a calibration position and the time delays to the three pulse receivers are measured. For i=1, 2, 3, 4, 5, 6 and 2, 3:

t_{i,j}=t_{dj}+t_{rec,j}+t_{tr,i}, (3)

where t_{rec,j} is the transmission cable delay and the processing time delay of the j-th receiver, and t_{tr,i} is the transmission cable delay of the pulse emitters. When the six pulse emitters are placed in sequence, the nine unknowns can be found from the eighteen equations using the method of least squared errors.

Once the delays t_{rec,j} and t_{tr,i} are determined, the true propagation time from any position to the pulse receivers can be found as follows:

t_{dj}=t_{i,j}−t_{rec,j}−t_{tr,i} (4)

In order to calculate the positions of the pulse emitters, the center point O of the reference axes is given by x₀, y₀, z₀. The coordinates of the pulse emitters 26, 28, 30 (receiving pulses R₁, R₂, R₃) are given by (0,0, μ); (0, μ, 0); and (μ, 0,0), respectively. The transmission signals are given as T₁, T₂, . . . Tₙ, and the position of the i-th transmitter emitting signal Tᵢ is given by equation set (5) below:

T_{d}(1,i)*c=d₁=\sqrt{(μ−xᵢ)²+yᵢ²+zᵢ²}

T_{d}(2,i)*c=d₂=\sqrt{xᵢ²+(μ−yᵢ)²+zᵢ²}

T_{d}(3,i)*c=d₃=\sqrt{xᵢ²+yᵢ²+(μ−zᵢ)²} (5)

where the solution of these equations can be obtained explicitly as follows:

$$x = \frac{- B + \sqrt{{B}^{2} - 12 C}}{6} ,$$

where B=2(α_{z}−α_{y}−μ); C=μ²+α_{y}²+α_{z}²−d₁²; and expressions for y and z are given as equation set (6) below:

$$y = x - {α}_{y} ; {α}_{y} = \frac{{d}_{2}^{2} - {d}_{1}^{2}}{2 μ} \\ z = x + {α}_{z} ; {α}_{z} = \frac{{d}_{1}^{2} - {d}_{3}^{2}}{2 μ} \qquad ( 6 )$$

and repeating the above equations for the six pulse emitters determines the coordinates of the positions of the six pulse emitters relative to the reference frame.

The orientation and position of the instrument I can be found from the location of its three pulse emitters 14, 16, 18. Assuming that these emitters may be represented in terms of their signals, T₁, T₂, and T₃, then we define the axes of the sensor body as iₛ, jₛ and kₛ. The origin of these axes is given as Oₛ. The position of Oₛ with respect to R₀ is given by:

Oₛ=T₁+(T₂−T₁)/2=[xₛ⁰, yₛ⁰, zₛ⁰], (7)

and the sensor axes are defined as

$${i}_{s} = \frac{( {T}_{2} - {O}_{s} )}{\vert ( {T}_{2} - {O}_{s} ) \vert}$$

and

$${j}_{s} = \frac{( {T}_{3} - {O}_{s} )}{\vert ( {T}_{3} - {O}_{s} ) \vert} ,$$

where kₛ is determined by the cross-product of iₛ and jₛ.

The homogenous transformation matrix of the sensor with respect to R₀ is given by:

$${}_{}^{{R}_{0}}{T}{}_{S}^{} = [ \begin{matrix} & & & {x}_{s}^{o} \\ & {R}_{3 \times 3} & & {y}_{s}^{0} \\ & & & {z}_{s}^{0} \\ 0 & 0 & 0 & 1\end{matrix} ] , \qquad ( 8 )$$

where the columns of the rotational matrix are the vectors iₛ, jₛ and kₛ, respectively,

The rotational angles for yaw (i.e., rotation about kₛ), roll (i.e., rotation about jₛ), and pitch (i.e., rotation about iₛ) of the handheld instrument I can then be found from the rotational matrix, and are given below as equation set (9):

$$Yaw = {φ}_{s} = {Cos}^{- 1} ( \frac{{R}_{1 , 1}}{\sqrt{1 - {R}_{3 , 1}^{2}}} ) \\ Pitch = {θ}_{s} = {Sin}^{- 1} ( \frac{{R}_{3 , 2}}{\sqrt{1 - {R}_{3 , 1}^{2}}} ) \\ Roll = {ψ}_{s} = - {Sin}^{- 1} ( {R}_{3 , 1} ) . \qquad ( 9 )$$

FIG. 11 illustrates the graphical user interface 40. The graphical user interface 40 displays a graphical representation of the patient's various body parts, such that the medical professional can select the body part to be tested. The system then displays an illustration of the selected body part and suggests preferred locations for the patient pulse emitter sets 34, 36 and the corresponding positions or orientation of the body axes. The medical professional will then position the patient pulse emitters on the patient's body, as shown by the graphical user interface 40.

In the following, the body axis will be generated from the location of three transmitters T₄, T₅, and T₆. The default origin O_{b} is chosen to be at the point of intersection of the normal from T₆ (the y_{b} axis in emitter set 34) on the line joining T₄ and T₅ (the x_{b} axis) in FIG. 1. Letting i_{b}=(T₅−T₄)/|T₅−T₄|, then O_{b}=T₄+((T₆−T₄)·i_{b}) i_{b} and j_{b}=(T₆−O_{b})/|T₆−O_{b})|. The direction of the k_{b} axis is determined by the cross-product of i_{b} and j_{b}.

The homogeneous transformation matrix with respect to R₀ is then given by:

$${}_{}^{{R}_{0}}{T}{}_{b}^{} = [ \begin{matrix} & & & {x}_{b}^{0} \\ & {R}_{3 \times 3} & & {y}_{b}^{0} \\ & & & {z}_{b}^{0} \\ 0 & 0 & 0 & 1\end{matrix} ] , \qquad ( 10 )$$

where the columns of the rotational matrix are the vectors i_{b}, j_{b} and k_{b}, respectively. The user may choose to rotate the body axis, or even create his or her own virtual axis, provided that the location of the virtual axis is defined with respect the default body axis.

If the measurement involves two or more body parts or if the measurement is related to a joint between body parts, it would then be preferable to establish an independent body axis at these parts. The system then utilizes additional patient pulse emitters (at least three more PBs) for each additional body axis. Once the medical professional selects a first body part and places the PBs and marks their positions on the display, the medical professional can then proceed to select another body part and install additional PEs. The system then proceeds in executing similar steps to identify the location of the additions PEs and calculates the location of the body axis.

The system can also track the position of the second set of axes with respect to the first set of axes, and the user can choose between selecting image/data to be registered with respect to the any of the axes or can choose automatic selection. For the addition of three additional PEs on another part of the patient's body, the transformation matrix of the second set of axes can be determined using similar computational steps to those described above.

Letting ^{R}^{₀}T_{b1} be the homogenous transformation matrix of the first set of body axes, and letting ^{R}^{₀}Tₕ₂ be the homogenous transformation matrix of the second set of body axes, then the position of the second set relative to the first set is given by:

[^{b}^{₁}T_{b}_{₂}]=[^{b}^{₁}T_{R}_{₀}][^{R}^{₀}T_{b1}]=[^{R}^{₀}T_{b}_{₁}]⁻¹[^{R}^{₀}T_{b}_{₂}]. (11)

The system will then automatically determine the new orientation and position of the second set of axes with respect to the first set of axes, and can immediately display the measurements performed with respect to the :first body axis, with respect to the second set of axes. Compensation of breathing can also be performed with respect to the inhalation position, exhalation position or an average value.

In order to determine the position of the instrument I with respect to the body axis, the instrument tip (or some other point of interest) is represented as d with respect to the sensor body origin O. Particularly if the instrument is a sensor, such a determination is not only of great interest, but must also have great accuracy. The position of the sensor with respect to origin O may be given as, for example, Pₛ=(0, d, 0, 1). Then, the position with respect to the body is given by P=[^{R}^{₀}Tₛ]Pₛ=[^{R}^{₀}T_{b}]P_{b}, or P_{b}=[^{R}^{_{b}}T_{R}_{₀}][[^{R}^{₀}Tₛ]Pₛ=[^{R}^{₀}T_{R}_{_{b}}]⁻¹[^{R}^{₀}Tₛ]Pₛ. This expression can be reduced to the following linear equation:

$$[ \begin{matrix}{x}_{b} \\ {y}_{b} \\ {z}_{b}\end{matrix} ] = {[ {}_{}^{{R}_{0}}{T}{}_{b}^{} ]}^{- 1} [ \begin{matrix}{T}_{1 , 2}^{s} d + {x}_{s}^{o} \\ {T}_{2 , 2}^{s} d + {y}_{s}^{o} \\ {T}_{3 , 2}^{s} d + {z}_{s}^{o}\end{matrix} ] , \qquad ( 12 )$$

Thus, the position of the sensor tip with respect to the body axis can be exactly determined and recorded together with the measurement. Assuming that the sensor is not touching the body, then the aiming beam intersection with the body, given by the (x_{b}, y_{b}), (x_{b}, z_{b}), or (y_{b}, z_{b}) planes, can also be determined. For example, the intersection with the (x_{b}, y_{b}) plane can be determined as follows:

^{R}_{₀}P_{b}=[^{R}_{₀}Tₛ][0Lₛ0 1]=[^{R}_{₀}T_{b}][x_{b} y_{b} 0 1]. (13)

Equation (13) is solved to obtain the intersection point (x_{b}, y_{b}, 0) in the patient's body. The intersection point will be highlighted on the graphical user interface of the display 38. This is given by the solution to the equation:

$$[ \begin{matrix}{T}_{1 , 1}^{b} & {T}_{1 , 2}^{b} & {T}_{1 , 2}^{s} \\ {T}_{2 , 1}^{b} & {T}_{2 , 2}^{b} & {T}_{2 , 2}^{s} \\ {T}_{3 , 1}^{b} & {T}_{3 , 2}^{b} & {T}_{3 , 2}^{s}\end{matrix} ] [ \begin{matrix}{x}_{b} \\ {y}_{b} \\ {L}_{s}\end{matrix} ] = [ \begin{matrix}{x}_{s}^{o} - {x}_{b}^{o} \\ {y}_{s}^{o} - {y}_{b}^{o} \\ {z}_{s}^{o} - {z}_{b}^{o}\end{matrix} ] . \qquad ( 14 )$$

If the body part is not moving (e.g., the patient P is under anesthesia), then a touching probe may be used to touch selected points on the limb or other body part to establish reference points. The points will be registered in the database 84 and displayed on the display 38. Then, a default body axis will be established and displayed on the display 38 in the same manner as described above with regard to the attached PEs.

It is to be understood that the present invention is not limited to the embodiments described above, but encompasses any and all embodiments within the scope of the following claims.

1. A method for tracking the position of handheld medical instruments, comprising the steps of:
providing a graphical user interface for displaying data related to at least a portion of a patient's body;
selecting a body part of the patient for performing a selected medical procedure;
establishing a plurality of pulse receivers for detecting and receiving very narrow pulse electromagnetic pulses;
mounting a plurality of instrument pulse emitters on a handheld medical instrument for use external to the patient's body for selectively transmitting first very narrow pulse electromagnetic pulses;
positioning a plurality of patient pulse emitters on the selected body part of the patient for selectively transmitting second very narrow pulse electromagnetic pulses;
determining a position of the handheld medical instrument with respect to the plurality of pulse receivers based upon travel time between transmission of the first very narrow pulse electromagnetic pulses and detection thereof;
determining a position of the selected body part with respect to the plurality of pulse receivers based upon travel time between transmission of the second very narrow pulse electromagnetic pulses and detection thereof;
determining a position of the handheld medical instrument with respect to the selected body part based upon the position of the handheld medical instrument with respect to the plurality of pulse receivers and the position of the selected body part with respect to the plurality of pulse receivers; and
establishing a default axis of the patient's body;
establishing at least one virtual axis of the patient's body;
selecting an axis of the patient's body.,
determining an orientation of the handheld medical instrument with respect to the selected axis of the patient's body; and
providing user feedback via the graphical user interface based upon the selected medical procedure and the position of the handheld medical instrument with respect to the selected body part, along with the orientation of the handheld medical instrument with respect to the selected axis of the patient's body.

2. The method for tracking the position of handheld medical instruments as recited in claim 1, wherein said step of determining the position of the handheld medical instrument with respect to the plurality of pulse receivers based upon travel time between transmission of the first very narrow pulse electromagnetic pulses and detection thereof includes the step of correcting for cable delay, said method further comprising the steps of course correlation and fine registration correlation.

3. The method for tracking the position of handheld medical instruments as recited in claim 2, wherein said step of determining the position of the handheld medical instrument with respect to the plurality of pulse receivers based upon travel time between transmission of the first very narrow pulse electromagnetic pulses and detection thereof further includes the step of correcting for processing time.

4. The method for tracking the position of handheld medical instruments as recited in claim 3, wherein said step of determining the position of the selected body part with respect to the plurality of pulse receivers based upon travel time between transmission of the second very narrow pulse electromagnetic pulses and detection thereof includes the step of correcting for cable delay.

5. The method for tracking the position of handheld medical instruments as recited in claim 4, wherein said step of determining the position of the selected body part with respect to the plurality of pulse receivers based upon travel time between transmission of the second very narrow pulse electromagnetic pulses and detection thereof includes the step of correcting for processing time.

6. The method for tracking the position of handheld medical instruments as recited in claim 5, further comprising the step of determining orientation of the handheld medical instrument with respect to the plurality of pulse receivers based upon travel time between transmission of the first very narrow pulse electromagnetic pulses from individual ones of the instrument pulse emitters and detection thereof.

7. The method for tracking the position of handheld medical instruments as recited in claim 6, further comprising the step of determining orientation of the selected body part with respect to the plurality of pulse receivers based upon travel time between transmission of the second very narrow pulse electromagnetic pulses from individual ones of the patient pulse emitters and detection thereof.

8. A method for tracking the position of handheld medical instruments, comprising the steps of:
providing a graphical user interface for displaying data related to at least a portion of a patient's body;
selecting a body part of the patient for performing a selected medical procedure;
establishing a plurality of pulse receivers for detecting and receiving very narrow pulse electromagnetic pulses;
mounting a plurality of instrument pulse emitters on a handheld medical instrument for use external to the patient's body for selectively transmitting first very narrow pulse electromagnetic pulses;
positioning a plurality of patient pulse emitters on the selected body part of the patient for selectively transmitting second very narrow pulse electromagnetic pulses;
determining position and orientation of the handheld medical instrument with respect to the plurality of pulse receivers based upon travel time between transmission of the first very narrow pulse electromagnetic pulses from individual ones of the instrument pulse emitters and detection thereof;
determining position and orientation of the selected body part with respect to the plurality of pulse receivers based upon travel time between transmission of the second very narrow pulse electromagnetic pulses from individual ones of the patient pulse emitters and detection thereof;
determining position and orientation of the handheld medical instrument with respect to the selected body part based upon the position and orientation of the handheld medical instrument with respect to the plurality of pulse receivers and the position and orientation of the selected body part with respect to the plurality of pulse receivers; and
establishing a default axis of the patient's body;
establishing at least one virtual axis of the patient's body;
selecting an axis of the patient's body;
determining an orientation of the handheld medical instrument with respect to the selected axis of the patient's body; and
providing user feedback via the graphical user interface based upon the selected medical procedure and the position of the handheld medical instrument with respect to the selected body part, along with the orientation of the handheld medical instrument with respect to the selected axis of the patient's body.

9. The method for tracking the position of handheld medical instruments as recited in claim 8, wherein said step of determining the position and orientation of the handheld medical instrument with respect to the plurality of pulse receivers based upon travel time between transmission of the first very narrow pulse electromagnetic pulses and detection thereof includes the step of correcting for cable delay, said method further comprising the steps of course correlation and fine registration correlation.

10. The method for tracking the position of handheld medical instruments as recited in claim 9, wherein said step of determining the position and orientation of the handheld medical instrument with respect to the plurality of pulse receivers based upon travel time between transmission of the first very narrow pulse electromagnetic pulses and detection thereof further includes the step of correcting for processing time.

11. The method for tracking the position of handheld medical instruments as recited in claim 10, wherein said step of determining the position and orientation of the selected body part with respect to the plurality of pulse receivers based upon travel time between transmission of the second very narrow pulse electromagnetic pulses and detection thereof includes the step of correcting for cable delay.

12. The method for tracking the position of handheld medical instruments as recited in claim 11, wherein said step of determining the position and orientation of the selected body part with respect to the plurality of pulse receivers based upon travel time between transmission of the second very narrow pulse electromagnetic pulses and detection thereof includes the step of correcting for processing time.

13-20. (canceled)
